(19) United States
(12) Patent Application Publication
(10) Pub. No.: US 2016/0279037 A1
(43) Pub. Date: Sep. 29, 2016

(54) COSMETIC COMPOSITIONS COMPRISING MICRO-SILVER AND METHODS OF APPLYING THE SAME

(71) Applicant: Sarkli-Repechage, Ltd., Secaucus, NJ (US)

(72) Inventor: Lydia Sarfati, New York, NY (US)

(73) Assignee: Sarkli-Repechage, Ltd., Secaucus, NJ (US)

(21) Appl. No.: 15/082,879

(22) Filed: Mar. 28, 2016

Related U.S. Application Data
(60) Provisional application No. 62/139,491, filed on Mar. 27, 2015.

(51) Int. Cl. A61K 8/19; A61Q 19/10; A61H 15/02; A61K 8/02; A61Q 1/02; A61K 8/98; A61K 8/97; A61Q 19/00

(52) U.S. Cl. CPC A61K 8/19; A61K 8/975; A61K 8/97; A61Q 19/10; A61Q 19/007; A61K 8/0216; A61K 8/0208; A61Q 1/02; A61K 8/987; A61H 15/02; A61K 2800/884; A61K 2800/70; A61K 2800/596

(57) ABSTRACT

The present disclosure relates to cosmetic compositions comprising micro-silver, kits comprising one or more of the cosmetic compositions, and methods for applying the same.

### CROSS-REFERENCE TO RELATED APPLICATIONS

This application claims the benefit under 35 USC §119(e) of U.S. Provisional Application Ser. No. 62/139,491, filed on Mar. 27, 2015, the entire disclosure of which is incorporated herein by reference

### BACKGROUND

Sensitive skin is a cosmetic condition that is often characterized by skin bumps, dry skin, flaky skin, and/or skin flushing. Symptoms of sensitive skin also include rashes, blotchiness, redness, itchiness, or stinging of the skin. Sensitive skin may be a result of skin damage due to environmental factors such as sun, wind, excessive heat or cold, or an allergic reaction. Sensitive skin is also commonly a symptom of a dermatological skin disorder such as eczema, rosacea, or dermatitis.

Rosacea, for example, is a chronic skin condition that manifests in small red bumps and flushed, red skin. Rosacea is often misdiagnosed as sunburn or acne. Several factors can contribute to the skin condition of rosacea such as genetic predisposition, immune response, and the skin's reaction to microbes present on the skin surface. Other factors can aggravate rosacea, such as exposure to intense sunlight, drinking alcohol, or eating dairy or spicy foods. Emotions, such as intense anger, embarrassment, or stress, can also aggravate rosacea. Left untreated, rosacea can worsen to create continual flushed, red skin over a subject's face, forehead, cheeks, chin, neck, nose, or anywhere on a subject's body. As such, optimal treatments of sensitive skin conditions, such as rosacea, are needed.

This disclosure is directed to address this problem and particularly relates to compositions and methods for treating sensitive skin conditions. More specifically, this disclosure relates to cosmetic and/or beauty-care compositions comprising micro-silver and methods of applying said compositions in order to treat sensitive skin conditions, such as rosacea. The resulting compositions and methods enable care and/or treatment of cosmetic skin conditions, such as sensitive skin and rosacea.

### SUMMARY OF THE INVENTION

The present disclosure is directed to a cosmetic composition for treatment of skin of a subject. The composition of the present disclosure comprises micro-silver, seaweed, and plant extract. Micro-silver may comprise from about 0.01% to about 1% of the weight of the cosmetic composition. Seaweed may comprise from about 0.05% to about 10% of the weight of the cosmetic composition, while plant extract may comprise from about 1% to about 25% of the weight of the cosmetic composition.

Seaweed of the present cosmetic composition may be selected from a group consisting of Laminaria Digitata, Ahnfeltia Concinna, Enteromorpha Compressa, and Ulva Lactuca. Plant extract of the present composition may be selected from the group consisting of Aloe Barbadensis, Mirabilis Jalapa, Rosmarinus Officinalis, Salvia Officinalis, Chamomilla Recutita, Hamamelis Viginiana, Cocos Nucifera, and Melissa Officinalis.

An exemplary cosmetic composition of the present disclosure is a serum composition, a cleanser composition, or an actives composition. The actives composition of the present disclosure may further comprise phyto-marine actives. In addition, the micro-silver of the cosmetic composition of the present disclosure comprises microparticles.

A kit for treatment of skin of a subject comprising the cosmetic composition is also disclosed herein. The kit may comprise one or more of the cosmetic compositions of the present disclosure, which may be housed in a unidose package. The kit may further comprise a toner composition, a cream composition, or one or more eye pads. The one or more eye pads may be impregnated with a liquid composition comprising seaweed and/or plant extract.

Finally, a method of applying the cosmetic compositions of the present disclosure is described herein. The application method of the present disclosure comprises applying and then removing a cleanser composition from the skin of a subject, applying and then removing a serum composition from the skin of a subject, and/or applying and then removing an actives composition from the skin of a subject, wherein the cleanser composition, the serum composition, and the actives composition each comprise micro-silver.

The step of applying and then removing a serum composition may further comprise massaging the skin of the subject that is performed manually or using a silver ball. Massaging the skin of the subject using the silver ball may comprise steps selected from the group consisting of: 1) slide and roll, 2) zygo slide, 3) full glide, 4) rolling zig zag, 5) smooth wave, 6) lifting slide, 7) lymph nodes, 8) neck strokes, 9) circle flow, 10) expanding waltz, 11) orbital glide, and 12) temporal area pressure points. The silver ball of the present method may comprise a ball component and a handle. The silver ball of the present method may also be made of silver, stainless steel, aluminum, or combinations thereof.

The application method of the present disclosure may further comprise mixing a gel composition and an actives product to form the actives composition, prior to applying and then removing the actives composition from the skin of the subject. The application method may also comprise mixing a gel composition, an actives product, and a powder composition comprising phyto-marine actives to form the actives composition, prior to applying and then removing the actives composition from the skin of the subject.

### BRIEF DESCRIPTION OF THE DRAWINGS

A brief description of the drawings is as follows:

FIG. 1 is a schematic of the exterior packaging of a commercial bottle embodiment of a cleanser composition of the present disclosure.

FIG. 2 is another schematic of the exterior packaging of the commercial bottle of the cleanser composition of FIG. 1.

FIG. 3 is a schematic of the unidose packaging of a commercial embodiment of the cleanser composition comprised in a kit of the present disclosure.

FIG. 4 is a schematic of the exterior packaging of a commercial bottle embodiment of a serum composition of the present disclosure.

FIG. 5 is a schematic of the packaging of the commercial bottle embodiment of the serum of the present disclosure.

FIG. 6 is a schematic of the unidose packaging of a commercial embodiment of the serum composition comprised in the kit of the present disclosure.

FIG. 7 is a schematic of the unidose packaging of a commercial embodiment of a mask powder composition comprised in the kit of the present disclosure.

FIG. 8 is a schematic of the unidose packaging of a commercial embodiment of a gel composition comprised in the kit of the present disclosure.

FIG. 9 is a schematic of the unidose packaging of a commercial embodiment of an actives product comprised in the kit of the present disclosure.

FIG. 10 is a schematic of the unidose packaging of a commercial embodiment of a toner/tonic composition comprised in the kit of the present disclosure.

FIG. 11 is a schematic of the unidose packaging of a commercial embodiment of a cream composition comprised in the kit of the present disclosure.

FIG. 12 is a schematic of the packaging of a commercial embodiment of an eye pad impregnated with a liquid composition and comprised in the kit of the present disclosure.

FIG. 13A shows the perspective view of a silver ball of the present disclosure.

FIG. 13B shows the side elevation view of the silver ball of the present disclosure.

FIG. 13C shows the front elevation view of the silver ball of the present disclosure.

FIG. 13D shows the rear elevation view of the silver ball of the present disclosure.

FIG. 14 is a schematic of the packaging and contents of a commercial embodiment of the kit of the present disclosure.

FIG. 15A is a schematic of the exterior packaging of the commercial kit embodiment of FIG. 14.

FIG. 15B is a schematic of the interior packaging of the commercial kit embodiment of FIG. 14.

### DETAILED DESCRIPTION OF THE INVENTION

The present disclosure is directed to cosmetic compositions and methods for care of skin and cosmetic treatment of sensitive skin or skin conditions of a subject. For example, sensitive skin is a condition that is commonly associated with a dermatological skin disorder such as eczema, rosacea, dermatitis, or acne. Exemplary skin conditions of the present disclosure are rosacea and acne.

Rosacea is a chronic skin condition that manifests in small red bumps and flushed, red skin. Acne is the most common skin condition in the U.S., which occurs due to clogged pores in skin and may be aggravated by microorganisms that infect the clogged pores causing inflammation. Further, the present disclosure is directed to reducing and/or slowing the effect of environmental aggressors on skin of a subject. Illustrative environmental aggressors of the present disclosure include, but are not limited to smog, air pollution, ultraviolet (UV) rays (e.g., UV A and B rays), extreme hot or cold temperatures, and more.

For the purpose of the present disclosure, “skin” comprises the forehead, cheeks, chin, neck, nose, ears, eyelids, hands, feet, fingers, arms, legs, back, shoulders, or any area of exposed skin on the body or person of a subject. A subject of the present disclosure may comprise a human subject, including an adult (over 18 years old) or an adolescent (under 18 years old). A subject of the present disclosure may be a customer or client of a cosmetic or beauty-care professional, such as a licensed esthetician. A subject of the present disclosure may also be a patient under care by a licensed physician (e.g., a dermatologist).

### COMPOSITIONS AND COMPONENTS OF THE PRESENT DISCLOSURE

Illustrative embodiments of the composition of the present disclosure include, but are not limited to, compositions comprising micro-silver, seaweed, and plant extract. The composition may also comprise actives, such as phyto-marine actives. An exemplary embodiment of the present disclosure is a serum composition, a cleanser composition, and an actives composition for the care of skin and sensitive skin.

Compositions of the present disclosure, including the serum composition, the cleanser composition, and the actives composition, comprise micro-silver. Micro-silver is produced from pure metallic silver, and is used as an active ingredient in the compositions of the present disclosure for care of normal, irritated, or inflamed skin, such as sensitive skin. In exemplary embodiments of the present disclosure, a micro-silver composition comprises, consists of, or consists essentially of metallic silver ranging from about 99.5% to about 100%, from about 99.6% to about 100%, from about 99.7% to about 100%, from about 99.8% to about 100%, and from about 99.9% to about 100% of pure silver. An illustrative embodiment of micro-silver of the present disclosure comprises about 99.7%, about 99.8%, about 99.9%, or about 100% of pure silver.

Micro-silver may be used in the present disclosure in the form of a liquid. However, an illustrative embodiment of micro-silver of the present disclosure comprises a metallic silver powder. The micro-silver powder comprises highly porous micro-sized particles.

The micro-silver powder granule may comprise about 1 to about 5 or more microparticles, but typically about one microparticle per micro-silver powder granule. The microparticles of the micro-silver powder may have a size that ranges from about 5 micrometers (μM) to about 15 μM, from about 6 μM to about 14 μM, from about 7 μM to about 13 μM, from about 8 μM to about 12 μM, from about 9 μM to about 11 μM, and at or about 10 μM in size.

The micro-sized particles of the micro-silver powder provide effective and long-lasting broad spectrum action (e.g., Gram +VE/−VE). In addition, the porous structure of the micro-silver powder provides efficient silver ion generation at low usage levels. Further, the porous structure of the micro-silver powder helps the micro-silver to physically attach and/or “cling” to the surface of the skin due to the microparticles. Therefore, the porous micro-silver comprising the microparticles results in natural, skin-friendly, safe, and long-lasting antimicrobial action on the surface of the skin of a subject.

The present disclosure is directed to cosmetic compositions comprising micro-silver, wherein micro-silver comprises, consists of, or consists essentially of about 0.01% to about 1% of the weight of the composition, from about 0.01% to about 0.75%, from about 0.01% to about 0.5%, from about 0.05% to about 0.5%, from about 0.05% to about 0.25%, from about 0.1% to about 0.5%, or from about 0.05% to about 0.2% of the weight of the composition. More specifically, an embodiment of the cleanser composition of the present disclosure may comprise from about 0.01% to about 0.5% of micro-silver, and typically from about 0.05% to about 0.25% of micro-silver. In addition, an embodiment of the serum composition of the present disclosure may comprise from about 0.05% to about 1% of micro-silver, and typically from about 0.1% to about 0.5% of micro-silver. Further, an embodiment of the actives composition of the present disclosure comprises from about 0.01% to about 0.5% of micro-silver, and typically from about 0.05% to about 0.2% of micro-silver.

While skin cells create free radicals in order to fight and neutralize microorganisms present on the surface of skin, environmental aggressors, such as pollution, can also spawn free radicals. Free radicals are very unstable, and gain stability by binding to a donor electron. In addition, free radicals contribute to premature aging of skin, such as wrinkles, fine lines, and age spots. Moreover, free radicals accelerate and/or irritate chronic skin conditions, such as rosacea and acne.

The compositions of the present disclosure are effective by releasing soluble silver ions at the skin's surface. The micro-silver provides an invisible layer of pure metallic silver that protects the surface of the skin against free radicals. The silver ions released at the skin's surface produce an antimicrobial effect by reacting to unwanted microorganisms causing them to be destroyed. Microorganisms that are susceptible to the silver ions of the micro-silver component of the compositions of the present disclosure include, but are not limited to, bacteria, viruses, fungi, germs, and other pathogenic microbes.

The silver ions kill microorganisms via several mechanisms, including inhibition of transmembrane transport proteins, inactivation of intracellular enzymes, and damage to microorganism DNA such that the microorganism cannot reproduce. Thus, the presence of silver ions on the skin's surface enables continuous generation of antimicrobial silver ions (Ag+) to provide long-term protection of the skin's surface. In fact, clinical studies have shown that use of micro-silver helps soothe chronically inflamed and irritated skin by killing bacteria that is used as a food source by skin irritating microbes.

The compositions of the present disclosure may also comprise kelp, which are large seaweeds. Seaweed has no roots, and feeds through osmosis, allowing it to absorb minerals and vitamins from the seawater. Through its feeding process, seaweed comprises a dense concentration of vitamins, minerals, and amino acids. Vitamins, such as beta-carotene, biotin, folic acid, B1, B2, B3, B5, B6, B12, C, E, and K1 have all been found to be present in seaweed. Nitrogen, phosphorus, potassium, sulfur, magnesium, and calcium are minerals that have also been found to be present in seaweed. Additionally, amino acids, such as alanine, arginine, aspartic acid, cysteine, glutamic acid, glycine, histidine, isoleucine, leucine, lysine, methionine, phenylalanine, proline, serine, threonine, tryptophan, tyrosine, and valine have also been found to be present in seaweed. Thus, seaweed is an optimal ingredient to calm or balance sensitive skin conditions.

The present compositions utilize various types of seaweed to provide optimal skin care benefit. In some compositions, seaweed may comprise from about 0.05% to about 10%, from about 0.2% to about 10%, or from about 0.5% to about 5% of the weight of the composition. More specifically, an embodiment of the cleanser composition of the present disclosure may comprise from about 0.5% to about 2% of seaweed, and typically from about 0.8% to about 1.5% of seaweed. In addition, an embodiment of the serum composition of the present disclosure may comprise from about 1% to about 5% of seaweed, and typically from about 2% to about 4% of seaweed. Further, an embodiment of the actives composition of the present disclosure comprises from about 0.05% to about 5% of seaweed, and typically from about 0.1% to about 2% of seaweed.

Types of seaweed of the present disclosure include, but are not limited to the following illustrative embodiments. Laminaria Digitata is a brown seaweed that is rich in antioxidants, helps hydrate and lock in moisture to the skin, and provides anti-bacterial and antimicrobial properties. Enteromorpha Compressa is a green seaweed that has soothing and anti-inflammatory properties Ahnfeltia Concinna is a red seaweed that helps soothe, hydrate, and nourishes skin, which ultimately helps to diminish the appearance of fine lines and wrinkles on the skin. Additionally, Ulva Lactuca is a green seaweed that has anti-inflammatory and antioxidant properties that help soothe skin.

Laminaria Digitata may comprise from about 0.01% to about 5% of weight of the composition. Enteromorpha Compressa may comprise from about 0.01% to about 1% of the weight of the composition. Ahnfeltia Concinna may comprise from about 0.1% to about 3% of the weight of the composition. Ulva Lactuca may comprise from about 0.005% to about 1% of the weight of the composition.

Actives are mineral sources and/or extracts from one or more plants, including, but not limited to seaweed, that provide benefits to skin when applied. For example, the phyto-marine actives of the compositions of the present disclosure may be obtained from seaweed and/or other plants. In the present compositions, phyto-marine actives may be in a powder formulation (i.e., “powder”) that comprises about 85% to about 100%, and typically from about 87% to about 95% of seaweed extracts and/or mineral sources. In addition, the illustrative actives composition of the present disclosure, which may comprise phyto-marine actives, provide benefits to skin, such as soothing, calming, and/or healing skin.

In addition to seaweed and actives, total plant extracts may comprise from about 0.1% to about 25%, from about 1% to about 25%, from about 3% to about 25%, and from about 1% to about 22% of the weight of the compositions. More specifically, an embodiment of the cleanser composition of the present disclosure may comprise from about 1% to about 15% of plant extracts, and typically from about 2% to about 12% of plant extracts. In addition, an embodiment of the serum composition of the present disclosure may comprise from about 1% to about 15% of plant extracts, and typically from about 3% to about 13% of plant extracts. Further, an embodiment of the actives composition of the present disclosure comprises from about 5% to about 25% of plant extracts, and typically from about 11% to about 24% of plant extracts.

Plant extracts include, but are not limited to, extracts from plants, fruits, nuts, spices, and vegetables, are also comprised in the present composition. Plant and fruit extracts have been shown in clinical studies to have properties that are beneficial to skin, including soothing irritation and inflamed skin, good skin bioaffinity, alleviating skin discomfort and redness, increasing skin resistance and hydration, restoration of skin collagen and elasticity, anti-inflammatory, antimicrobial, antifungal, antibacterial, and astringent properties.

While the plant extracts may be in organic forms or inorganic forms, preferably, plant extracts are organic. Any addition of a denaturant or unnatural component to an organic composition or extract transforms the organic compositions into an inorganic composition. In addition, the plant extracts may comprise any form including, but not limited to, powders, pastes, gels, oils, waxes, or liquids, such as juice or water. Extracts may also be obtained from any portion of the plant or fruit including the fruit, bark, leaves, peel, seeds, roots, stem, flower, thistle, thorn, or any other plant part.

Illustrative embodiments of plants from which extracts are comprised in the present composition include, but are not limited to, Aloe Barbadensis (i.e., Aloe Vera), Mirabilis Jalapa, Rosmarinus Officinalis (i.e., Rosemary), Salvia Officinalis (i.e., Sage), Chamomilla Recutita (i.e., Matricaria), Hamamelis Viginiana (i.e., Witch Hazell), and Melissa Officinalis (i.e., Lemon Balm). Individual plant extracts may range from about 0.10% to about 30% of a composition of the present disclosure. Specific embodiments of fruit plants from which extracts are comprised in the present composition include, but are not limited to citrus plants (e.g., oranges, lemons, limes, grapefruits, etc.) or other fruit plants such as coconuts (i.e., Cocos Nucifera), apples, bananas, etc. Extracts from spices, such as turmeric, may also be used in the present composition.

The compositions of the present invention may also comprise an aqueous component or solution including, but not limited to water, whether deionized water or sterile water. The aqueous component of the present disclosure may comprise from about 50% to about 95%, from about 50% to about 90%, from about 50% to about 80%, from about 60% to about 80%, and from about 55% to about 75% of the weight of the compositions. More specifically, an embodiment of the cleanser composition of the present disclosure may comprise from about 50% to about 75% of aqueous composition, and typically from about 55% to about 70% of aqueous composition. In addition, embodiments of the serum composition and the actives composition of the present disclosure may comprise from about 50% to about 95% of aqueous composition, and typically from about 60% to about 80% of aqueous composition.

The compositions of the present disclosure may comprise a multitude of additional components including, flavonoids, proteins (i.e., silk and wheat proteins), trace elements, antioxidants, sugars, surfactants, solvents, salts, preservatives, buffers, emollients, emulsifiers, thickeners, binders, foam boosters, viscosity increasing or decreasing agents, waxes, preservatives, emulsion stabilizers, or perfumes or aromas. Notably, aromas of the present disclosure only comprise natural scents, and do not comprise any chemical fragrances.

Quercetin and rutin are phenolic compounds found in plants. Illustrative embodiments of antioxidants of the subject invention comprise quercetin and rutin, which help combat free radicals that are damaging to skin. In addition, quercetin and rutin may also be comprised in the present invention as flavonoids to reduce free radicals and other environmental aggressors or stressors. Typically, quercetin comprises from about 0.005% to about 0.5%, from about 0.01% to about 0.25%, or from about 0.01% to about 0.2% of the weight of the compositions of the present disclosure. More specifically, an embodiment of the cleanser composition of the present disclosure may comprise from about 0.005% to about 0.25% of quercetin, and typically from about 0.01% to about 0.15% of quercetin. In addition, embodiments of the serum composition and the actives composition of the present disclosure may comprise from about 0.005% to about 0.15% of quercetin, and typically from about 0.01% to about 0.1% of quercetin.

In addition, rutin may comprise from about 0.05% to about 1%, from about 0.05% to about 0.6%, or from about 0.1% to about 0.5% of the weight of the compositions of the present disclosure. More specifically, an embodiment of the cleanser composition of the present disclosure may comprise from about 0.05% to about 1% of rutin, and typically from about 0.1% to about 0.5% of rutin. In addition, embodiments of the serum composition and the actives composition of the present disclosure may comprise from about 0.05% to about 0.5% of rutin, and typically from about 0.1% to about 0.3% of rutin. In addition, hyaluronic acid has a strong affinity to skin and increases skin resilience and hydration. Hyaluronic acid may also be comprised in the present compositions.

Viscosity decreasing agents of the compositions of the present disclosure include, but are not limited to, butylene glycol, propanediol, and combinations thereof. Total solvents or viscosity decreasing agents may be comprised in the present compositions at a weight percent ranging from about 0.05% to about 15%. More specifically, an embodiment of the cleanser composition of the present disclosure may comprise from about 0.05% to about 5% of viscosity decreasing agents, and typically from about 1% to about 5% of viscosity decreasing agents. In addition, an embodiment of the serum composition of the present disclosure may comprise from about 1% to about 10% of viscosity decreasing agents, and typically from about 2% to about 8% of viscosity decreasing agents. Further, an embodiment of the actives composition of the present disclosure comprises from about 5% to about 15% of viscosity decreasing agents, and typically from about 6% to about 12% of viscosity decreasing agents.

Illustrative embodiments of viscosity increasing components of the present compositions include, but are not limited to, sodium acrylate/sodium acryloyl dimethyl taurate copolymers and neopentyl glycol dicaprylate (dicapriate). Total viscosity increasing agents may be comprised in the present compositions at a weight percent ranging from about 0% to about 10%. More specifically, an embodiment of the cleanser composition of the present disclosure may comprise from about 2% to about 8% of viscosity increasing agents, and typically from about 4% to about 7% of viscosity increasing agents. In addition, an embodiment of the serum composition of the present disclosure may comprise from about 1% to about 7% of viscosity increasing agents, and typically from about 2% to about 4% of viscosity increasing agents. Further, an embodiment of the actives composition of the present disclosure comprises from about 0% to about 5% of viscosity increasing agents.

Surfactants, emollients, and/or emulsifiers may be comprised in the present compositions at a weight percent ranging from about 0% to about 35%. Surfactants, emollients, and/or emulsifiers that may be comprised in the present invention include, but are not limited to polyglyceryl-3 stearate, shea butter ethyl esters, squalene, tridecyl stearate, neopentyl glycol dicaprylate (dicaprate), sodium acrylate/sodium acryloyl dimethyl taurate copolymers, cetyl alcohol, steareth-10, ethyl macadamiate, stearyl alcohol, and cocamidopropyl betaine.

Preservatives may be comprised in the present compositions at a weight percent ranging from about 0.1% to about 5%. Illustrative preservatives, include, but are not limited to phenoxyethanol and ethylhexylglycerin. Sugars, polysaccharides, or sugar byproducts that may be comprised at a weight percent ranging from about 0.05% to about 1% in the present invention at include, but are not limited to, xanthan gum and rutin. Salts that may be comprised in the present composition include, but are not limited to, sodium hyaluronate and sea salt. Salts may comprise a weight percent ranging from about 0% to about 5% of the present composition.

A kit of the present disclosure comprises at least one, one or more, two or more, or all three of the serum composition, the cleanser composition, and the actives composition. For example one embodiment of the present kit may comprise only one composition, such as the serum composition, the cleanser composition, or the actives composition. Alternatively, the kit may comprise two of the compositions, or all three of the compositions. Illustrative embodiments of the formulation of the cleanser composition and the serum composition of the present disclosure are shown in Tables 1 and 2, respectively.

In addition, the kit of the present disclosure may comprise one or more additional components to help calm or soothe skin including, but not limited to, a gel, a powder, and an actives product. For example, the actives composition of the present disclosure may be comprised of the gel, the actives product, and/or the powder, and combinations thereof. These compositions may be used alone, mixed together, and mixed separately with other compositions in order to form different embodiments of the actives composition of the present disclosure.

Illustrative embodiments of a formulation of the gel composition and the actives product of the present disclosure are shown in Tables 3 and 4, respectively. An exemplary embodiment of the actives composition of the present disclosure is a mixture of the gel composition and the actives product, such as an 1:1 ratio, a 1:1.2 ratio, or a 1:1.5 ratio of the gel composition mixed with the actives product. For example, a mixture comprising a single unidose package of the gel composition and the actives product may form an embodiment of the present actives composition. A further embodiment of the actives composition of the present disclosure is a mixture of the powder, the gel composition, and the actives product. This embodiment of the actives composition may form an activated mask composition that is applied to the skin of a subject during the method of application described herein.

The kit of the present disclosure may comprise additional components, such as one or more of a tonic, a cream, and a pad. For example, a tonic of the present disclosure is an aqueous composition that may comprise seaweed, in addition to natural moisturizers, such as witch hazel and glycerin. An illustrative embodiment of the formulation of the tonic of the present disclosure is shown Table 5. A cream of the present disclosure may be for the protection of skin, as exemplified in the formulation of Table 6.

A pad may be an eye pad used to protect the eyes, sooth the eyes, and reduce puffiness of the eyes during skin treatment and/or application of compositions. An illustrative formulation of the eye pads of the present disclosure is shown in Table 7. Notably, the eye pads of the present disclosure may be presoaked in a liquid composition comprising seaweed and/or plant extract, such that the eye pads are impregnated with the seaweed and/or plant extract before being placed on eyes of a subject.

The kit of the present disclosure may also comprise instructions for use and/or application of the compositions, along with one or more utensil devices and/or small pieces of equipment to aid preparation and/or application of the present compositions to a subject's skin. For example, utensil devices of the present kit may comprise one or more of a band (e.g., for hair), a spatula, a ball, a hand wipe, a bowl, and cotton.

More specifically, a bowl of the present disclosure may be comprised of rubber. Rubber is a pliable material that may be of a medical grade similar to what is used in medical or dental offices for treatment of patients. The bowl comprising rubber or medical grade rubber conforms to the shape of the users hand, provides for less slippage, stronger grip, and thus, enables easier and more consistent mixing (e.g., with the spatula) of compositions of the present disclosure prior to application to a subject's skin.

In addition, one or more sanitary or antibacterial hand wipes may be comprised in the kit of the present invention. More specifically, about 1 to about 20, typically about 5 to about 10, and often about 5 sanitary hand wipes may be comprised in the kit of the present disclosure. Users may clean their hands with the self-contained and/or prepackaged hand wipes before initiating the method of applying the compositions of the present disclosure as described herein.

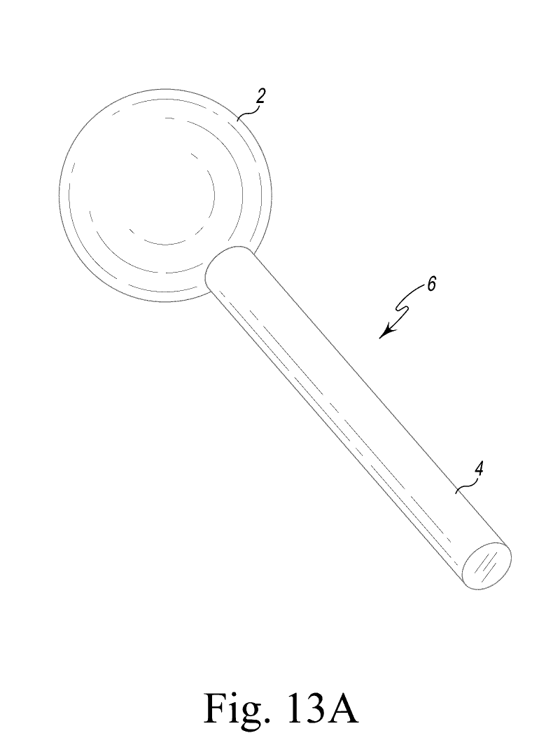

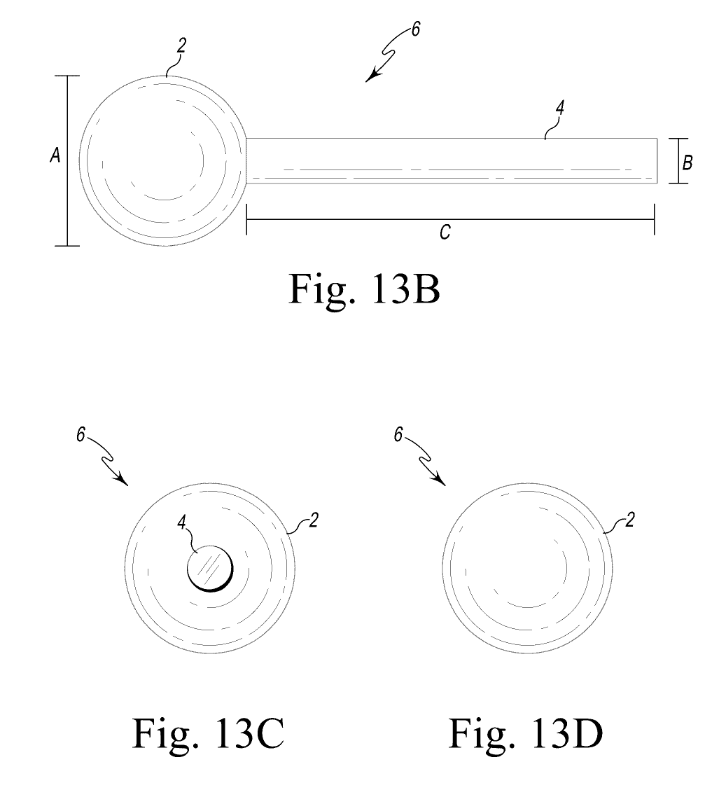

As shown in FIGS. 13A-13D, the ball (6) of the present disclosure may be used to massage compositions of the present disclosure into the skin of the subject and/or apply compositions onto the skin of the subject. The handle (4) of the ball (6) is manually held during application of compositions to skin of the subject. Typically, the proximal end of the ball component (2) of the ball (6) comes into contact with compositions and/or skin when the compositions are applied onto skin of the subject. For example, the ball (6) may be used to apply the serum composition to skin of the subject and to further massage the serum composition into the skin of the subject.

An illustrative embodiment of the ball (6) of the present disclosure comprises stainless steel, silver (Ag+), aluminum, or combinations thereof. An exemplary stainless steel comprised in the ball (6) is a 316 grade stainless steel, while an exemplary silver comprised in the ball is micro-silver. Typically, the ball (6) will comprise about 95% to about 100% of stainless steel silver, aluminum, or a combination thereof.

The weight of the solid ball (6), including the ball component (2) and the handle (4), ranges from about 130 grams to about 140 grams, from about 131 grams to about 138 grams, from about 132 grams to about 136 grams, from about 133 grams to about 135 grams, and typically weighs about 133 grams. The weight of the ball provides pressure when applying the compositions of the present disclosure onto the skin of the subject to enable proper and beneficial massage of the skin and muscles (e.g., facial muscles, see FIGS. 13A-13D and Example 10).

The ball also enables a glide massage with no friction, which is beneficial for the treatment of skin and/or skin conditions. For example, the ball remains consistently cool or cold during the massage of skin. This temperature modulation and/or reduction of skin by the ball are beneficial for improved skin care results. More specifically, subjects suffering from skin conditions, such as rosacea or sensitive skin, particularly benefit from use of the ball for application of skin treatments since heat or friction are harmful and further inflame the skin affected by skin conditions. The ball may or may not be comprised in the kit of the present disclosure.

The kit of the present disclosure is designed to allow a user, such as the licensed esthetician or physician, to easily reproduce the same results each time the contents of the kit are used according to the application method described herein. Contents of the kit, including the serum composition, the cleanser composition, and the actives composition, or a combination thereof may be comprised in a single dose amount, also called unidose packaging. Unidose packaging of the compositions of the present disclosure comprises the contents of each composition needed for only a single application.

Compositions of the kit of the present disclosure may comprise a unidose ranging from about 1.5 grams (g) to about 85 g, and typically ranges from about 5 g to about 10 g, from about 7 g to about 9 g, from about 6 g to about 8 g, and often between about 7 to about 8 grams. For example, a unidose of the serum composition comprises about 6 g to about 8 g, whereas a unidose of the cleanser composition and the actives product comprises from about 7 g to about 9 g of each composition. However, a unidose of the compositions of the present disclosure may also be larger or small. For example, a unidose of the mask comprises from about 20 g to about 22.5 g, and typically about 21 g of the powder composition. A unidose of the gel composition ranges from about 70 g to about 85 g, from about 75 g to about 81 g, and typically about 79 g. A unidose of the cream composition ranges from about 1 g to about 5 g, from about 1.5 g to about 2.5 g, and typically about 2 g.

Unidose packaging (e.g., a packette) of the presently described kit compositions provides beneficial skin care results to the subject. Notably, the compositions may be prepared, mixed, and applied to skin of a subject more consistently and accurately since a premeasured and prepackaged amount of each composition formulation has been provided in the kit. Unidose packaging also provides ease of use of kit contents since no measuring is required. In addition, unidose packaging enables a user to better monitor and control inventory since one can much easier determine how many treatments have been used and/or applied to subjects. Importantly, unidose packaging of kit contents provides for better health and safety of the skin of the subject because use of a fresh, unopened product during each application reduces the risk of contamination of a single product due to repeated uses of a single product on different subjects. The risk of cross-contamination of other products by a single product is also mitigated with unidose packaging.

### METHODS OF APPLYING THE COMPOSITIONS OF THE PRESENT DISCLOSURE

A method of applying one or more compositions of the present disclosure is also disclosed herein. The method comprises manual and/or machine application of one or more compositions of the present disclosure to the subject. Typically, the method described herein is manually performed by a skin care professional. For example, a skin care professional includes, but is not limited to, a licensed esthetician or a licensed physician.

In one embodiment, the method comprises application of one or more of the serum composition, the cleanser composition, and the actives composition, or a combination thereof. In another embodiment, the method may comprise removal of one or more of the serum composition, the cleanser composition, and the actives composition, or a combination thereof. In a further embodiment, the method comprises application and removal of one or more of the serum composition, the cleanser composition, and the actives composition, or a combination thereof. The standard time to complete the application method ranges from about 30 minutes to about 1 hour, from about 40 minutes to about 1 hour, from about 45 minutes to about 55 minutes, and at or about 40 minutes, 45 minutes, 50 minutes, 55 minutes, or 1 hour.

In addition, the method of applying and/or removing compositions of the present disclosure may comprise one or several steps and/or substeps (i.e., steps). In particular, the method of the present disclosure may comprise 1 step, 2 steps, 3 steps, 4 steps, 5 steps, 6 steps, 7 steps, 8 steps, 9 steps, 10 steps, 11 steps 12 steps, 13 steps, and preferably about 5 steps to about 8 steps. For example, an illustrative embodiment of the present method comprises the following application and removal steps:

Step 1—Cleanser

Step 2—Toner

Step 3—Serum

Step 4-4A—Powder; 4B—Gel; 4C—Actives; Eye Pads

Step 5—Protection Cream

More specifically, an illustrative embodiment of the present method comprises the following application and removal steps and/or substeps:

Step 1: Cleanse skin. Apply contents of cleanser packette into palm of hands and apply to skin using circular motions. Gently remove cleanser with cotton saturated in lukewarm water (i.e., water temperature ranging from about 30° C. to about 40° C.).

Step 2: Thoroughly mist skin with toner or apply toner lightly to skin with cotton. Pat dry gently with tissue or cotton.

Step 3: Cut open packette of serum and gently apply to skin. Massage serum into skin for about 5 minutes to about 7 minutes. The massage may be performed manually with one's hands or using a silver ball. For example, a user may perform light effleurage movements to massage subject's skin to help apply serum onto and/or into the skin. Effleurage massage movements, which are light, continuous stroking movements on the skin, improve blood circulation in blood vessels near the surface of the skin. Accordingly, massaging skin using such technique improves poor blood circulation, which reduces the presence of wrinkles, fine lines, and age spots, while improving overall skin tone.

Step 4: Apply an actives composition, such as an activated mask composition to skin of the subject. The mask may need preparation prior to application to skin as follows.

Substep 4A: Empty contents of mask powder packette into a rubber bowl.

Substep 4B: Empty contents of gel activator packette into the bowl with powder and mix thoroughly with spatula until smooth.

Substep 4C: Add in contents of actives product and blend into powder/gel mixture with spatula until homogenous to finalize the actives composition in the form of an activated mask composition. Alternatively, the gel and actives product contents of Substeps 4B and 4C, respectively, may be mixed together and the contents of the mask powder of Substep 4A may be added to the gel/actives mixture and stirred until smooth.

Substep 4D: Apply activated mask composition to skin with spatula. Cover eyes with eye pads and leave mask on for about 10 minutes to about 15 minutes. Gently remove mask by lifting it from the skin and folding it over. Remove any mask residue gently with wet cotton.

Step 5: Empty contents of protection cream packette into hands and apply on skin gently in upward and outward strokes.

The method of applying one or more compositions of the present disclosure onto the skin of the subject is described in steps 1-5 above. An exemplary embodiment of the present method is described and further demonstrated in Example 9. Notably, the step of applying the serum composition to skin of the subject (i.e., step 3 above) may also comprise massage steps, which may be performed by manual massage using effleurage movements as previously described.

A silver ball of the present disclosure may also be used to massage the skin of the subject rather than massaging manually. For example, the technique of massaging the skin with the silver ball to apply a composition, such as the serum composition, may comprise from about 1 step to about 15 steps, from about 1 step to about 12 steps, from about 1 step to about 10 steps, from about 1 step to about 8 steps, from about 1 step to about 6 steps, from about 1 step to about 4 steps, and from about 1 step to about 2 steps. Exemplary steps of applying the compositions of the present disclosure to skin using the silver ball may comprise, consist of, or consist essentially of the following massage technique steps: 1) slide and roll, 2) zygo slide, 3) full glide, 4) rolling zig zag, 5) smooth wave, 6) lifting slide, 7) lymph nodes, 8) neck strokes, 9) circle flow, 10) expanding waltz, 11) orbital glide, and/or 12) temporal area pressure points (see Example 10). Massage with the silver ball provides all of benefits as that described herein for effleurage movements, with additional benefits, such as skin temperature reduction due to the cool temperature of the ball and limited to no friction to the skin.

In addition to steps 1-5 of the method of applying the present compositions as described above, the present method may also comprise steps that are optional. For example and preferably, a user may sanitize their hands prior to beginning the present application method. The skin may also be pre-cleaned prior to actually applying the cleanser composition of the present disclosure. Pre-existing make-up may be removed from the skin of the subject before beginning the application method and/or make-up may be applied to skin of the subject after the application method described herein is completed. During the approximate 10-15 minute wait time while the mask is on the skin of the subject, a massage of skin on other areas of the subject's body may be performed. For example, if the mask has been applied to the face of a subject, a massage of the hands and the arms of the subject may be performed by the user.

### EXAMPLES

Illustrative embodiments of the serum composition, the cleanser composition, the actives composition, the toner composition, the gel composition, the eye pad composition, and the cream composition of the present disclosure are provided herein by way of examples. It will be appreciated that the methods and compositions described herein have broad applications. The foregoing embodiments were chosen and described in order to illustrate principles of the compositions and methods as well as some practical applications. While certain embodiments have been described and/or exemplified above, it is contemplated that considerable variation and modification thereof are possible. The preceding description enables others skilled in the art to utilize methods and compositions in various embodiments and with various modifications as are suited to the particular use contemplated. In accordance with the provisions of the patent statutes, the principles and modes of operation of this disclosure have been explained and illustrated in exemplary embodiments. Accordingly, the present invention is not limited to the particular embodiments described and/or exemplified herein.

### Example 1

### Cleanser Composition

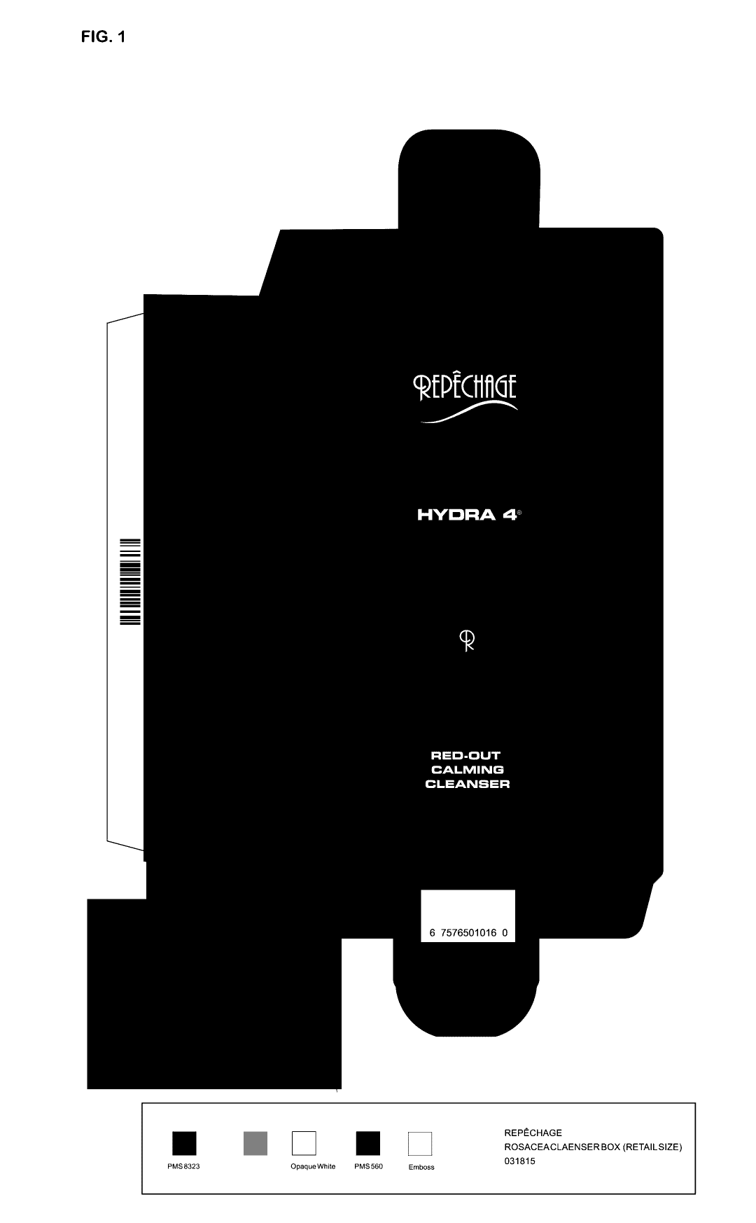

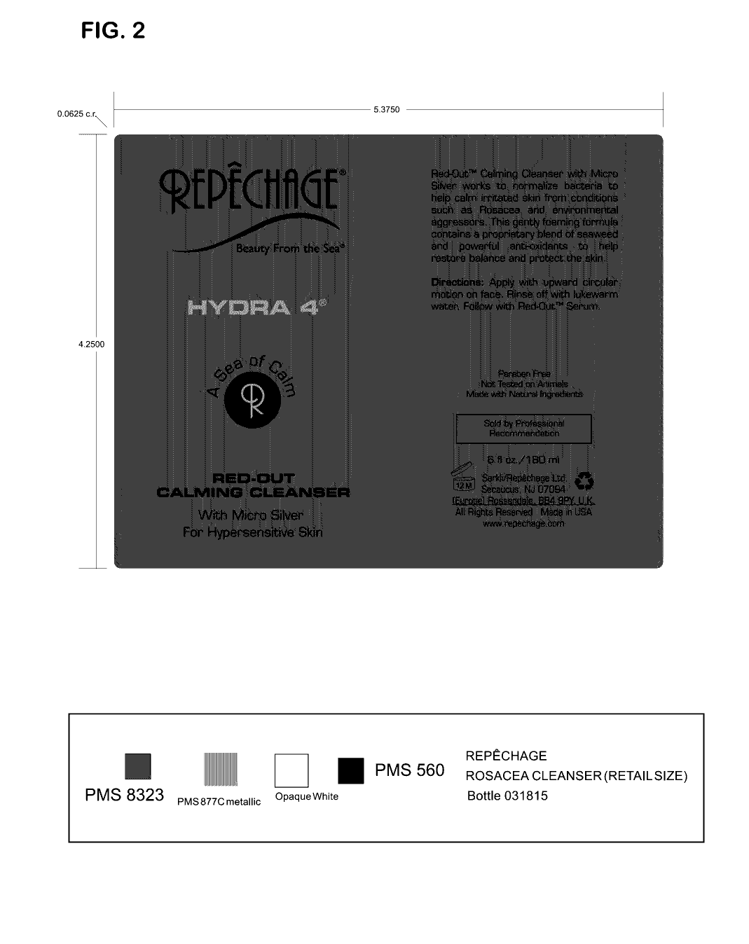

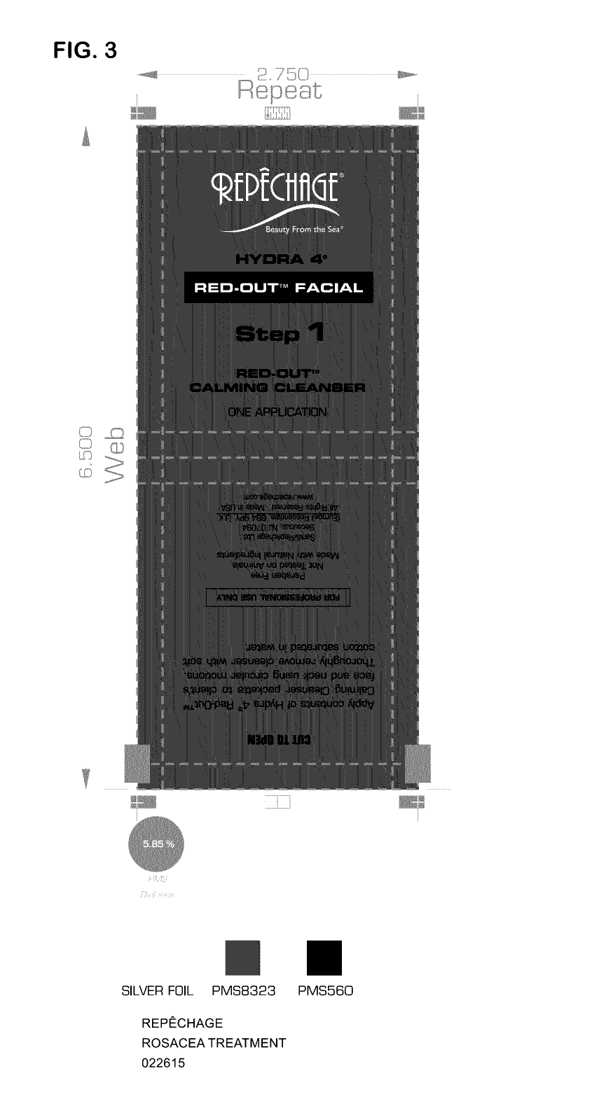

This example details the ingredients comprised in an illustrative embodiment of the wash/cleanser composition of the present disclosure. An example of the general formulation of the cleanser composition of the present disclosure, including micro-silver, is shown in Table 1. Moreover, an illustrative formulation of the cleanser composition of the present disclosure, including micro-silver, is shown in Table 8. The packaging for commercial embodiments of the cleanser composition of the present disclosure is also provided. For example, exterior packaging for a full-sized commercial embodiment of the cleanser composition is shown in FIGS. 1 and 2. In addition, the unidose packaging of the cleanser composition comprised in the kit of the present disclosure is shown in FIG. 3.

### Example 2

### Serum Composition

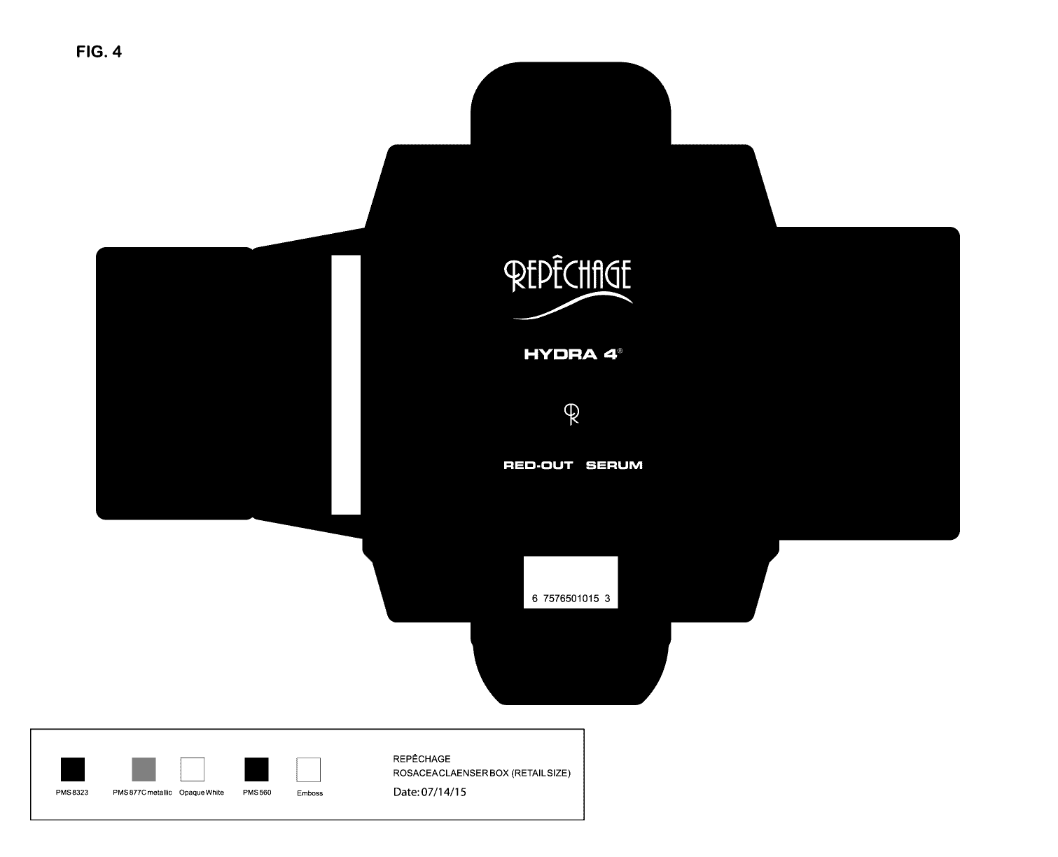

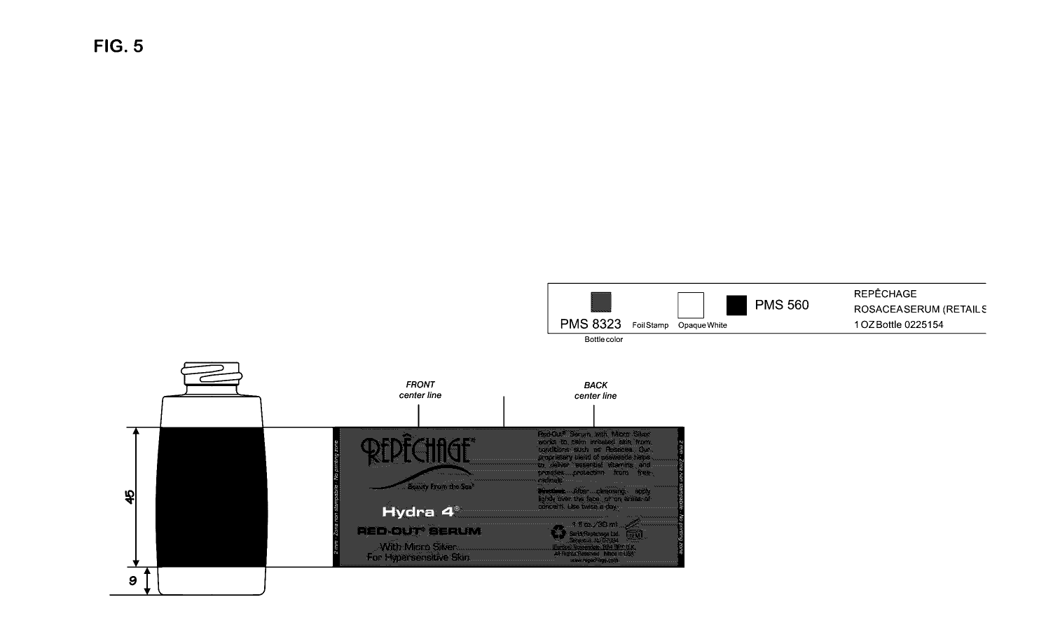

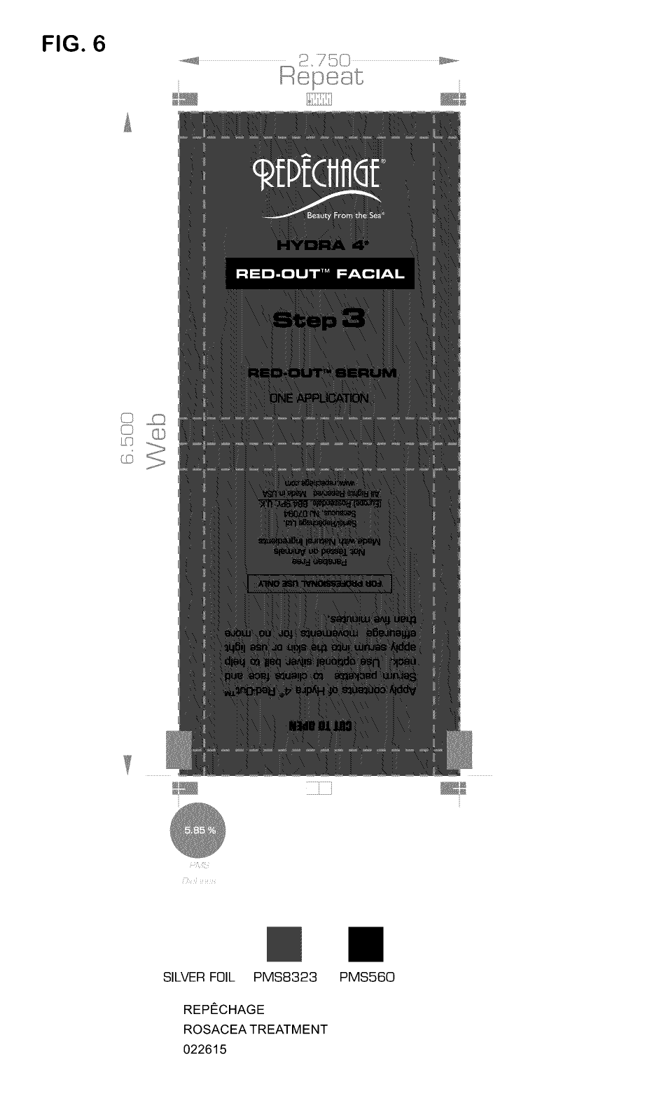

This example details the ingredients comprised in an illustrative embodiment of the serum composition of the present disclosure. An example of the general formulation of the serum composition of the present disclosure, including micro-silver, is shown in Table 2. Moreover, an illustrative formulation of the serum composition of the present disclosure, including micro-silver, is shown in Table 9. The packaging for commercial embodiments of the serum composition of the present disclosure is also provided. For example, exterior packaging for a full-sized commercial embodiment of the serum composition is shown in FIG. 4, while the packaging for the full-sized serum bottle is shown in FIG. 5. In addition, the unidose packaging of the serum composition comprised in the kit of the present disclosure is shown in FIG. 6.

### Example 3

### Actives Compositions

This example details the ingredients comprised in an illustrative embodiment of the actives product of the present disclosure. In addition to the actives product, the actives composition of the present disclosure may also comprise a gel and/or a powder both described in detail herein. More specifically, a general formulation of a gel composition and an actives product, which may be mixed to prepare an embodiment of the present actives composition, are shown in Tables 3 and 4, respectively. Moreover, an illustrative formulation of the gel composition and the actives product are shown in Table 10 and 11, respectively. Notably, the resulting actives composition comprises micro-silver.

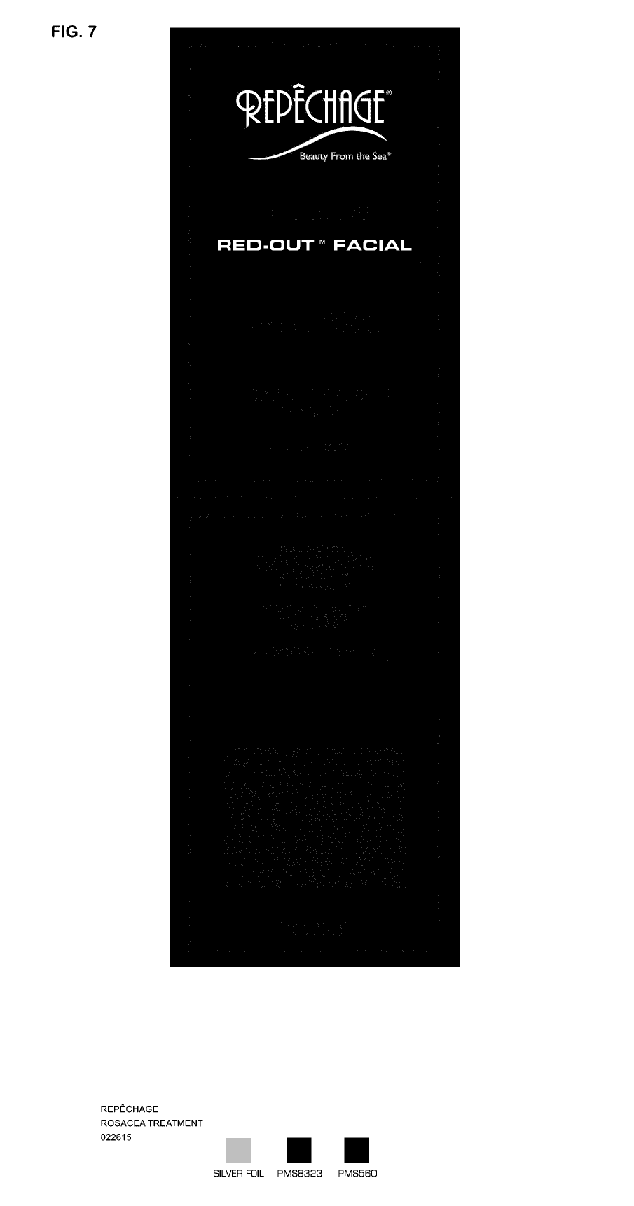

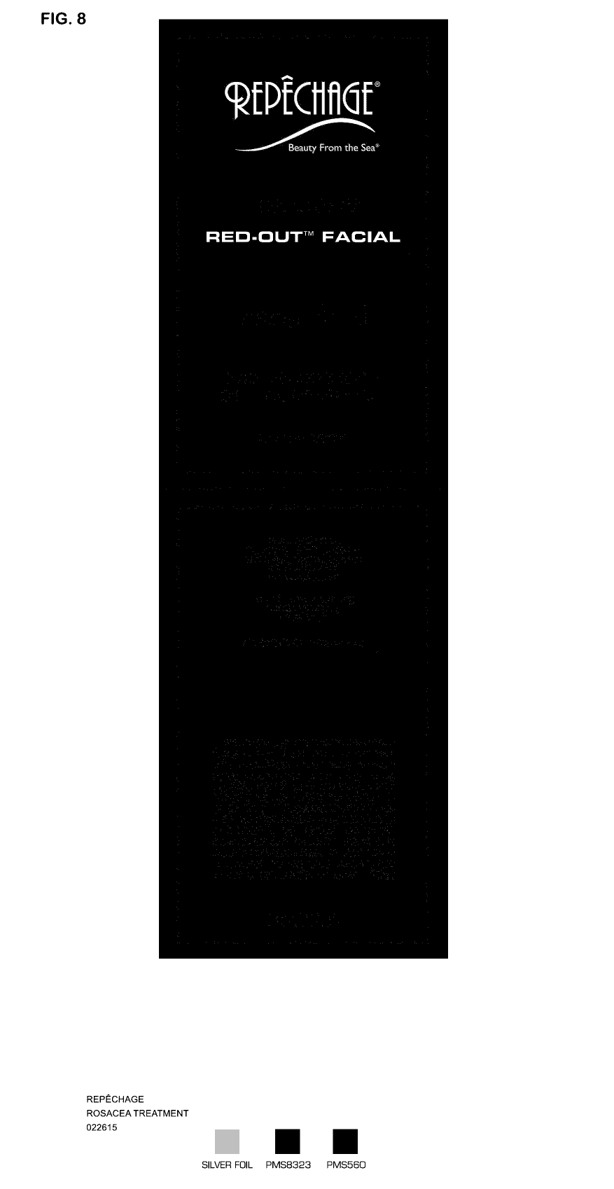

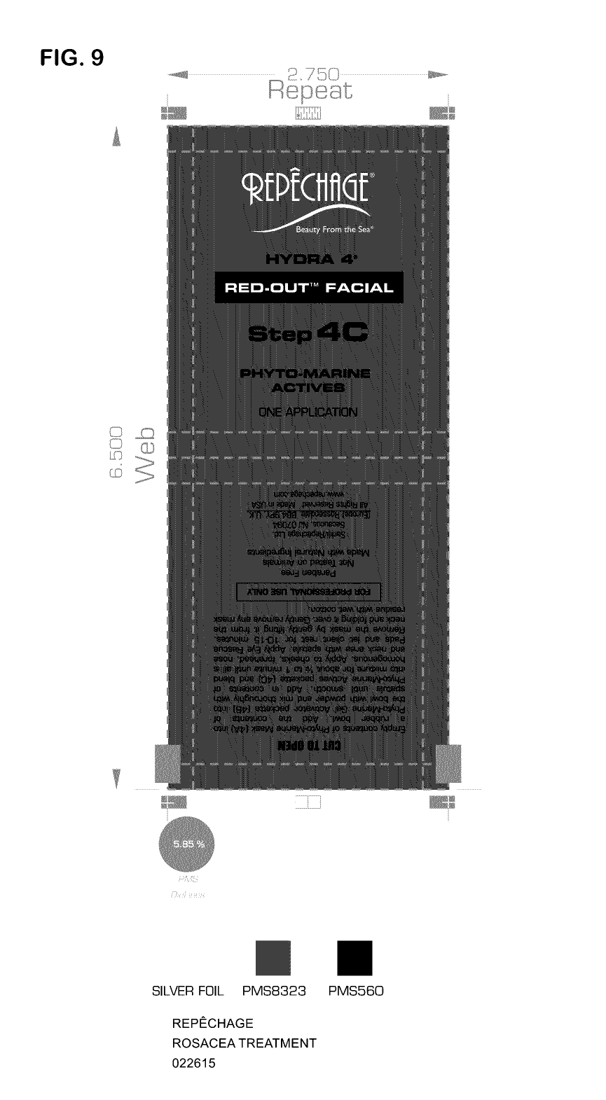

The powder may comprise actives, such as phyto-marine actives. Further, the powder may be added to the mixture of the gel composition and the actives product to prepare an additional embodiment of the actives composition of the present disclosure that forms an activated mask composition. The unidose packaging for a commercial embodiment of the powder, the gel, and the actives product of the kit of the present disclosure are shown in FIGS. 7, 8, and 9, respectively.

### Example 4

### Toner Composition

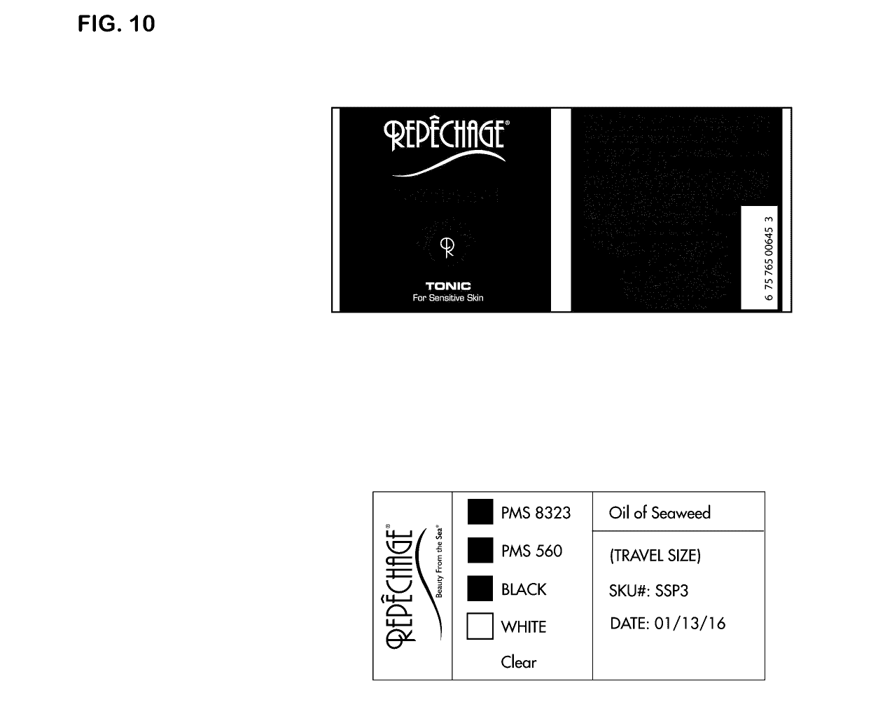

This example demonstrates an illustrative formulation of the toner composition of the present disclosure. An example of the general formulation of the toner composition of the present disclosure is shown in Table 5. Moreover, an illustrative formulation of the toner composition of the present disclosure is shown in Table 12. The unidose packaging for a commercial embodiment of the toner composition of the kit of the present disclosure is shown in FIG. 10.

### Example 5

### Cream Composition

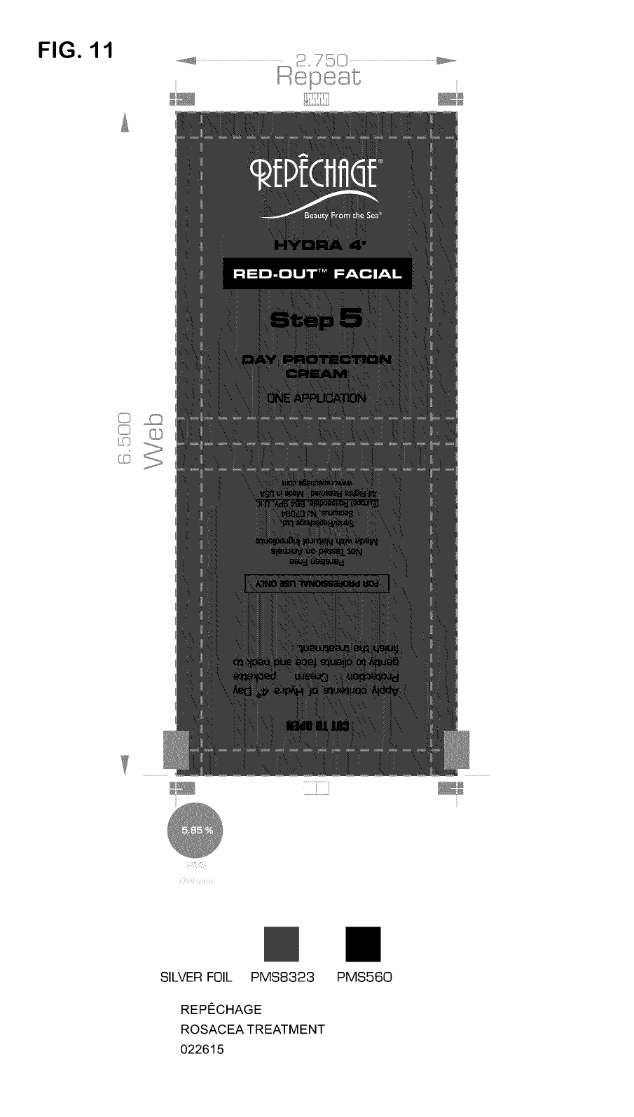

This example demonstrates an illustrative formulation of the cream composition of the present disclosure, wherein the cream may be a protection cream. An example of the general formulation of the cream composition of the present disclosure is shown in Table 6. Moreover, an illustrative formulation of the cream composition of the present disclosure is shown in Table 13. The unidose packaging for a commercial embodiment of the cream composition of the kit of the present disclosure is shown in FIG. 11.

### Example 6

### Pad Composition

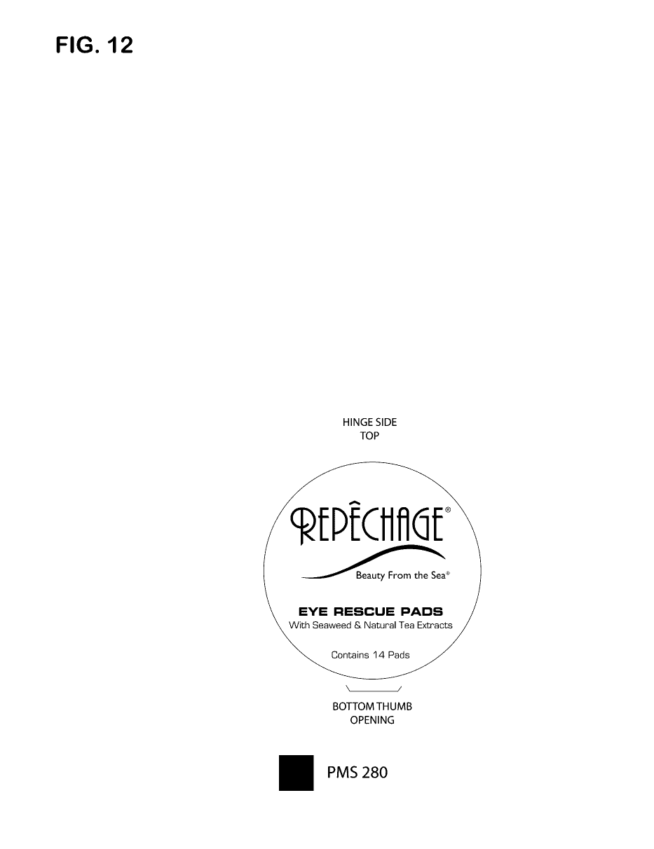

This example demonstrates an illustrative formulation of the pad composition of the present disclosure, wherein the pads may be eye pads. Notably, the eye pads are soaked in a liquid composition comprising seaweed and/or plant extract. An example of the general formulation of the eye pad composition of the present disclosure is shown in Table 7. Moreover, an illustrative formulation of the eye pad composition of the present disclosure is shown in Table 14. The packaging for a commercial embodiment of the pad composition of the present disclosure is shown in FIG. 12.

### Example 7

### Silver Ball

FIGS. 13A-13D shows an illustrative embodiment of the ball (6) used in the method of the present disclosure. The ball (6) of the present disclosure comprises a solid ball component (2) that is attached to a solid handle (4). More specifically, the distal end of the ball component (2) is attached to the proximal end of the handle (4). However, the proximal end of the ball component (2) and the distal end of the handle (4) are free and unattached (see FIGS. 13A-13B).

As seen in FIG. 13B, the ball component (2) of the ball (6) comprises a diameter (A) that ranges from about 1 inch to about 1¼ inches, and is typically about 1⅛ inches in diameter. The handle (4) of the ball (6) has a diameter (B) that ranges from about ¼ inch to about 6/16 inches long, and is typically about 5/16 inches in diameter. The handle (4) also has a length (C) of about 3 inches to about 3¼ inch in length, and is typically about 3 inches long.

### Example 8

### Facial Kit

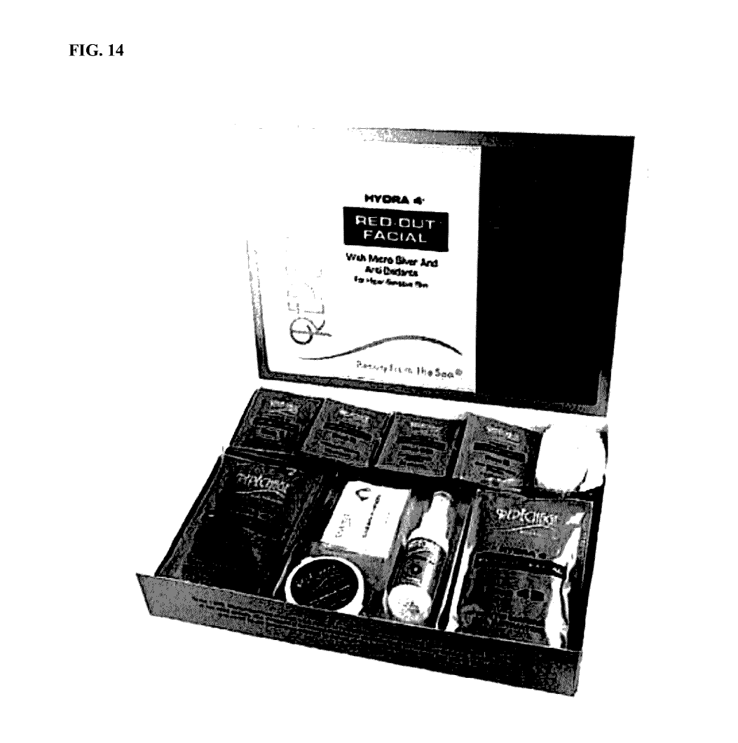

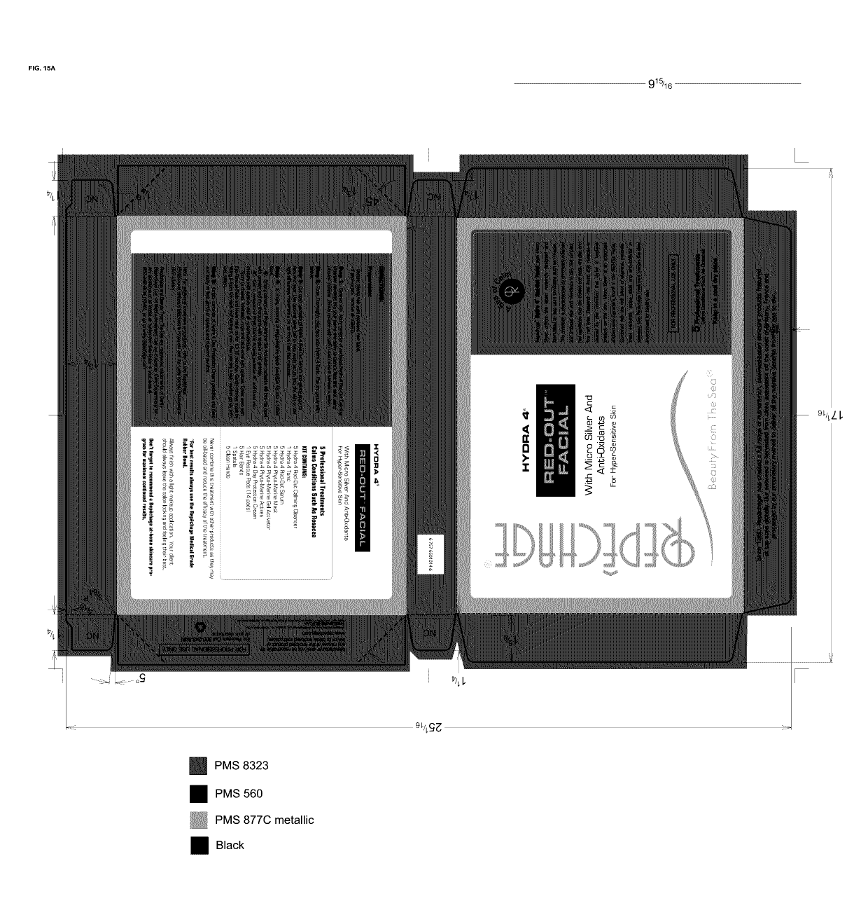

FIG. 14 demonstrates an illustrative commercial embodiment of the kit of the present disclosure. An exemplary embodiment of the present kit is a facial kit. An illustrative embodiment of the facial kit of the present disclosure comprises unidose packaging for most or all of the kit components. For example, the facial kit may comprise from about 1 to about 10, and typically from about 1 to about 5 unidose packages of each component, including, but not limited to the cleanser composition, the toner/tonic composition, the serum composition, the cream composition, the actives compositions and/or the components to prepare the actives composition, including the powder composition, the gel composition, and the actives product. Notably, the serum composition, the cleanser composition, and the actives composition comprise micro-silver.

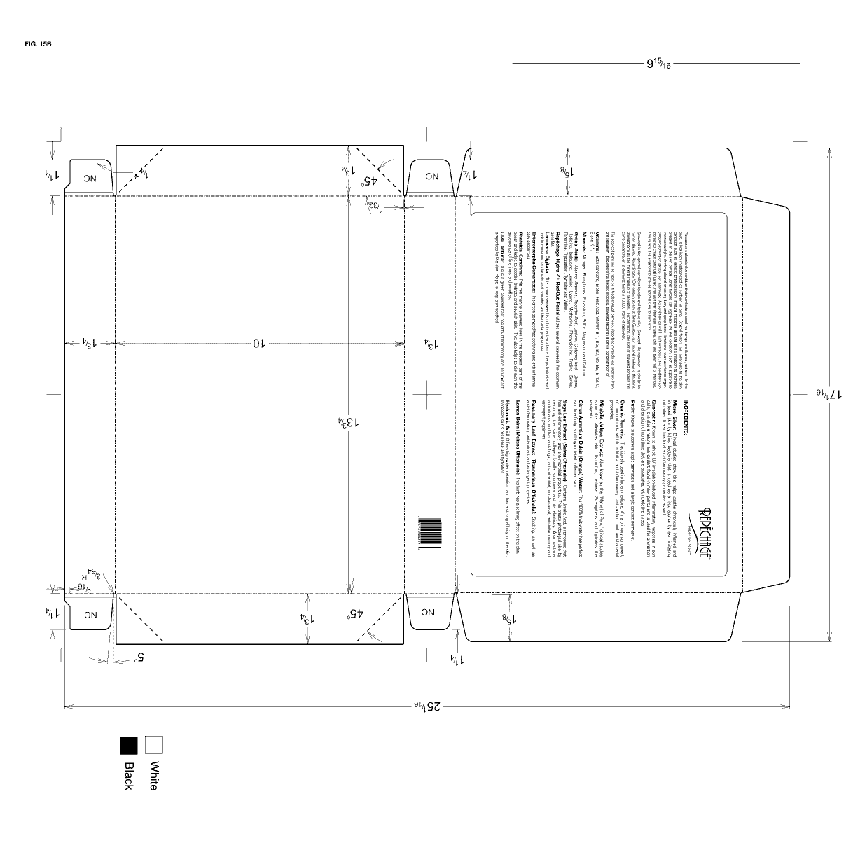

In addition, the illustrative facial kit may also comprise utensils to aid in preparation and application of the compositions which include, but are not limited to eye pads, hair bands, a spatula, and antibacterial hand wipes. The exterior and interior packaging for a commercial embodiment of the facial kit of the present disclosure is shown in FIGS. 15A and 15B, respectively.

### Example 9

### Method of Applying the Compositions

This example demonstrates an illustrative stepwise method of applying and removing the compositions of the present disclosure. A commercial method typically includes steps 1-5 as previously described in addition to optional steps such as, sanitizing hands, pre-cleaning the skin, and removing make-up prior to beginning the method. In addition, optional steps of massaging untreated skin of the body (e.g., hand and arm) and reapplication of make-up after the method is complete are also comprised in a commercial method embodiment.

### Example 10

### Massage Method Using the Silver Ball

This example demonstrates an illustrative stepwise method of massaging skin of a subject using the silver ball of the present disclosure. User holds the handle of the ball in their hand, such that the ball component of the ball comes in contact and applies pressure to the skin and muscles of the subject. Massage technique steps using the silver ball comprise the following: 1) slide and roll, 2) zygo slide, 3) full glide, 4) rolling zig zag, 5) smooth wave, 6) lifting slide, 7) lymph nodes, 8) neck strokes, 9) circle flow, 10) expanding waltz, 11) orbital glide, and/or 12) temporal area pressure points.

It is intended that the scope of the present compositions and methods be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

The scope of the disclosure should be determined, not with reference to the above description, but should instead be determined with reference to the appended claims, along with the full scope of equivalents to which such claims are entitled. It is anticipated and intended that future developments will occur in the arts discussed herein, and that the disclosed compositions and methods will be incorporated into such future examples. Furthermore, all terms used in the claims are intended to be given their broadest reasonable constructions and their ordinary meanings as understood by those skilled in the art unless an explicit indication to the contrary is made herein. In particular, use of the singular articles such as “a,” “the,” “said,” etc. should be read to recite one or more of the indicated elements unless a claim recites an explicit limitation to the contrary. It is intended that the following claims define the scope of the disclosure and that the method and apparatus within the scope of these claims and their equivalents be covered thereby. In sum, it should be understood that the disclosure is capable of modification and variation and is limited only by the following claims.

We claim:

1. A cosmetic composition for treatment of skin of a subject, comprising:
a) micro-silver,
b) seaweed, and
c) plant extract.

2. The cosmetic composition of claim 1, wherein the micro-silver comprises from about 0.01% to about 1% of the weight of the cosmetic composition.

3. The cosmetic composition of claim 1, wherein the seaweed comprises from about 0.05% to about 10% of the weight of the cosmetic composition.

4. The cosmetic composition of claim 1, wherein the plant extract comprises from about 1% to about 25% of the weight of the cosmetic composition.

5. The cosmetic composition of claim 1, wherein the seaweed is selected from the group consisting of Laminaria Digitata, Ahnfeltia Concinna, Enteromorpha Compress, and Ulva Lactuca.

6. The cosmetic composition of claim 1, wherein the plant extract is selected from the group consisting of Aloe Barbadensis, Mirabilis Jalapa, Rosmarinus Officinalis, Salvia Officinalis, Chamomilla Recutita, Hamamelis Viginiana, Cocos Nucifera, and Melissa Officinalis.

7. The cosmetic composition of claim 1, wherein the cosmetic composition is selected from the group consisting of a serum composition, a cleanser composition, or an actives composition.

8. The cosmetic composition of claim 7, wherein the actives composition further comprises phyto-marine actives.

9. The cosmetic composition of claim 1, wherein the micro-silver comprises microparticles.

10. A kit for treatment of skin of a subject, wherein the kit comprises one or more of the cosmetic compositions of claim 1.

11. The kit of claim 10, wherein the one or more cosmetic compositions of claim 1 are housed in a unidose package.

12. The kit of claim 10, wherein the kit further comprises a composition selected form the group consisting of a toner composition, a cream composition, and one or more eye pads.

13. The kit of claim 12, wherein the one or more eye pads are impregnated with a liquid composition comprising seaweed or plant extract.

14. A method of applying a cosmetic composition to skin of a subject, wherein the method comprises:
a. applying and then removing a cleanser composition from the skin of the subject,
b. applying and then removing a serum composition from the skin of the subject, or
c. applying and then removing an actives composition from the skin of the subject, wherein the cleanser composition, the serum composition, and the actives composition each comprise micro-silver.

15. The method of claim 14, wherein step (b) further comprises massaging the skin of the subject that is performed manually or using a silver ball.

16. The method of claim 15, wherein massaging the skin of the subject using the silver ball comprises steps selected from the group consisting of: 1) slide and roll, 2) zygo slide, 3) full glide, 4) rolling zig zag, 5) smooth wave, 6) lifting slide, 7) lymph nodes, 8) neck strokes, 9) circle flow, 10) expanding waltz, 11) orbital glide, and 12) temporal area pressure points.

17. The method of claim 15, wherein the silver ball comprises a ball component and a handle.

18. The method of claim 17, wherein the silver ball is made of silver, stainless steel, aluminum, or combinations thereof.

19. The method of claim 14, further comprising mixing a gel composition and an actives product to form the actives composition, prior to applying and then removing the actives composition from the skin of the subject.

20. The method of claim 14, further comprising mixing a gel composition, an actives product, and a powder composition comprising phyto-marine actives to form the actives composition, prior to applying and then removing the actives composition from the skin of the subject.
